(19) United States
(12) Patent Application Publication
(10) Pub. No.: US 2009/0247986 A1
(43) Pub. Date: Oct. 1, 2009

(54) Flapper Valve with Membrane Bypass for Power PICC

(76) Inventors: Robert F. Rioux, Ashland, MA (US); Kathleen Fernald-Kane, Brookline, MA (US); David Sauvageau, Methuen, MA (US)

Correspondence Address: FAY KAPLUN & MARCIN, LLP, 150 BROADWAY, SUITE 702, NEW YORK, NY, 10038, US

(21) Appl. No.: 12/334,934

(22) Filed: Dec. 15, 2008

Related U.S. Application Data
(60) Provisional application No. 61/014,959, filed on Dec. 19, 2007.

(51) Int. Cl. A61M 5/175

(52) U.S. Cl. 604/508; 604/256; 604/247

(57) ABSTRACT

A bypass for power injection includes (a) a housing defining a lumen extending from a proximal end to a distal end coupled to a catheter; (b) a membrane valve mounted within the lumen, the membrane valve having an opening extending therethrough sized to tightly receive therethrough and seal around a power injection device; and (c) a flapper valve extending across the lumen distally of the membrane valve. The flapper valve includes a flap biased toward a closed position in which the lumen is sealed. The flap is rotatable away from the closed position when contacted by a power injection device to permit the device to extend therethrough.

### PRIORITY CLAIM

This application claims the priority to the U.S. Provisional Application Ser. No. 61/014,959, entitled “FLAPPER VALVE WITH MEMBRANE BYPASS FOR POWER PICC,” filed Dec. 19, 2007. The specification of the above-identified application is incorporated herewith by reference.

### BACKGROUND

Procedures for the treatment of chronic diseases often require repeated and prolonged access to the vascular system. The more often these procedures are repeated, the more impractical and dangerous it becomes to insert and remove a needle at every session. Thus, patients requiring frequent sessions are often fitted with a semi-permanent catheter with a distal end opening into a vein and an accessible proximal end which is sealed when the catheter is not in use.

The proximal end is often sealed by a valve such as a Pressure Actuated Safety Valve (PASV) designed to open only when a fluid pressure in the catheter exceeds a preselected threshold pressure. PASV's often include a slitted membrane which flexes open when a fluid pressure above the threshold value is applied and which closes as soon as the pressure drops below the threshold to prevent fluid from leaking from the catheter and to prevent contaminants from entering therein.

The proximal end of the catheter may extend through the skin to remain accessible outside the body and may include provisions for connection to external devices. For example, a connector may be attached to the catheter, or may be formed at the proximal end of the catheter so that an external medical device may be placed in fluid connection thereto. These connectors may include the flow control valves (e.g., PASV's) described above in a housing to permit fluids to enter and/or exit the catheter only under predefined conditions. The flow control valve housing may either be part of the catheter or may be a separate component connected to the catheter body when the device is implanted. One type of such catheter is the peripherally inserted central catheter (PICC) which allows access to portions of the vascular system deep inside the body. A relatively long portion of the catheter is tunneled into the body while a proximal end remains accessible at a convenient location.

Most therapeutic procedures infuse fluids at a slow flow rate and a low injection pressure. For example, chemotherapy agents, drugs and blood products are typically delivered at low flow rates and pressures. In certain procedures, however, fluids are administered at higher pressures. For example, contrast media used in the visualization of blood vessels and other biological structures require special injection procedures including flow rates and pressures which are often higher than those which the flow control valves can withstand without damage. These procedures, commonly referred to as power injection procedures, typically require a separate, more robust catheter than those used for conventional infusion techniques.

### SUMMARY OF THE INVENTION

The present invention is directed to a bypass for power injection, comprising a housing defining a lumen extending from a proximal end to a distal end coupled to a catheter and a membrane valve mounted within the lumen, the membrane valve having an opening extending therethrough sized to tightly receive therethrough and seal around a power injection device in combination with a flapper valve extending across the lumen distally of the membrane valve, the flapper valve including a flap biased toward a closed position in which the lumen is sealed, the flap being rotatable away from the closed position when contacted by a power injection device to permit the device to extend therethrough.

### BRIEF DESCRIPTION OF DRAWINGS

FIG. 1 is a schematic diagram showing a bypass channel according to an embodiment of the present invention;

FIG. 2 is an illustration of a bypass membrane valve and a bypass flapper valve according to an embodiment of the invention;

FIG. 3 is an illustration of the bypass flapper valve shown in FIG. 2 in an open configuration; and

FIG. 4 is an illustration of the bypass flapper valve and the bypass membrane valve shown in FIG. 2, with a conduit passing therethrough.

### DETAILED DESCRIPTION

The present invention may be further understood with reference to the following description and the appended drawings, wherein like elements are referred to with the same reference numerals. The invention relates to devices used to connect a source of pressurized fluid to a valved catheter without damaging a valve of the catheter. More specifically, the invention relates to a bypass for a PICC catheter allowing power injections thereto without damaging a valve of the catheter and preventing back flow of fluids through the bypass.

Exemplary embodiments of the present invention provide for a catheter and, in particular, a bypass portion of a valved catheter enabling the catheter to be used for both low pressure infusions and power injections. The exemplary device prevents damage to the safety valve by employing a bypass lumen for power injections which isolates a valve of the catheter from the high pressures and flow rates of the power injections. The bypass lumen according to the present invention seals around the power injection device to prevent back flow and, when the bypass lumen is not in use, it is reliably sealed preventing leaks and contamination, even after repeated insertion of a power injection nozzle therethrough.

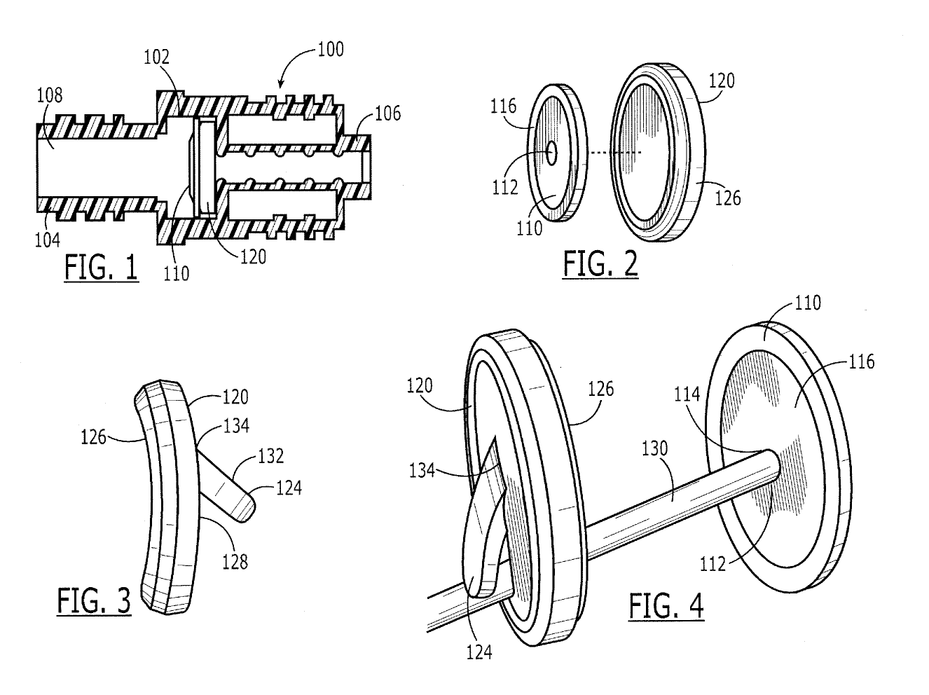

As shown in FIG. 1, a bypass 100 according to the invention defines a lumen 108 containing a sealing mechanism including a flapper valve 120 and a membrane valve 110 in series between proximal and distal ends 104, 106, respectively, of a housing 102. The proximal end 104 of the housing 102 may be open for the insertion of surgical instruments into the lumen 108 while the distal end 106 is coupled to a catheter (not shown) via, for example, a connector (not shown). The catheter would also extend proximally to a separate proximal port (not shown) for use in low pressure, low flow rate fluid infusions and withdrawals.

FIG. 2 shows the membrane valve 110 and the flapper valve 120 in greater detail. The membrane valve 110 comprises a flattened disk portion 116 attached to the housing 102 and extending substantially across the lumen 108. As would be understood by those skilled in the art, the disk portion 116 may be formed of a conventional polymer used for membrane valves or of any other suitable flexible material. An opening 112 is formed in the membrane valve 110 to permit passage therethrough of a nozzle, needle, syringe or other device used to perform the power injection procedure. The opening 112 is preferably shaped to correspond to an outer shape of the power injection device and may, for example, be circular with dimensions selected to permit insertion of the power injection device without tearing or otherwise damaging the membrane valve 110.

As shown in FIG. 3, the flapper valve 120 comprises a disk element 126 coupled within the housing 102 extending substantially across the lumen 108. The disk element 126 includes a flap 124 cut from a generally central portion thereof so that the flap 124 pivots relative to the rest of the flapper disk element along a hinge 134. Thus, the flap 124 may be pushed open as a device is advanced though the lumen 108. When the bypass 100 is not in use, the flap 124 is urged toward the closed position by, for example, a natural bias provided by the resilience of the material forming the disk element 126 and/or by additional biasing elements. The edge 132 of the flap 124 is preferably shaped to form a seal with a mating portion of the rest of the disk element 126 so that, as soon as a device is withdrawn from the bypass 100, the lumen 108 is sealed by the flapper valve 120. The flapper valve 120 may also preferably be formed so that it can only open toward the distal end 106 of the housing 102. This prevents fluid from flowing back into the bypass 100 when fluids are being infused or withdrawn through the low flow rate/low pressure infusion port.

As shown in FIG. 4, an exemplary device 130 passes along the lumen 108 through the membrane valve 110 and then through the flapper valve 120 which is distal thereto. In this embodiment, the membrane valve 110 is placed upstream, i.e. proximally from the flapper valve 120. As the device 130 passes through the opening 112 in the membrane valve 110, a sealing portion 114 of the disk portion 116 forms a seal around the outer surfaces of the medical device 130 preventing back flow therethrough. As the device 130 is further advanced through the lumen 108, it passes through the flapper valve 120 pushing the flap 124 open so that it pivots about the hinge 134. When inserted in this manner through the bypass 100, the power injection procedure may take place without leaks or back flow because even though the flapper valve 120 is open, the membrane valve 110 forms a seal around the device 130.

After completion of the power injection procedure and the device 130 is withdrawn from the lumen 108, the flap 124 of the flapper valve 120 closes and seals the lumen 108 and the membrane valve 110 is left with the opening 112, but is no longer necessary to close the bypass 100 because that function is now performed by the flapper valve 120. The low pressure/low flow rate port of the catheter may then be used in its conventional manner as the bypass 100 is sealed against back flow.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. A bypass for power injection, comprising:
a housing defining a lumen extending from a proximal end to a distal end coupled to a catheter;
a membrane valve mounted within the lumen, the membrane valve having an opening extending therethrough sized to tightly receive therethrough and seal around a power injection device; and
a flapper valve extending across the lumen distally of the membrane valve, the flapper valve including a flap biased toward a closed position in which the lumen is sealed, the flap being rotatable away from the closed position when contacted by a power injection device to permit the device to extend therethrough.

2. The bypass according to claim 1, wherein the flapper valve comprises a hinge pivotally coupling the flap to a mounting element coupling the flapper valve to the housing.

3. The bypass according to claim 1, wherein the flapper valve is formed as a check valve opening only toward the distal end of the housing.

4. The bypass according to claim 1, wherein the membrane valve substantially seals the lumen around a power injection device inserted therethrough.

5. The bypass according to claim 1, wherein the flapper valve is formed of a resilient material biasing the flap toward the closed position.

6. The bypass according to claim 1, wherein the flapper valve includes a biasing member biasing the flap toward the closed position.

7. A catheter system comprising:
a flexible elongate body including a distal portion insertable to a desired position within a body and a proximal portion which, when the distal end is inserted to the desired position within the body, remains accessible outside the body, the proximal portion including a bypass channel and an infusion channel, lumens of the bypass and infusion channels extending from proximal openings to distal openings opening into a catheter lumen extending to the distal portion of the catheter;
a valve extending across the infusion channel and sealing the channel at all times when a fluid pressure in the infusion channel remains below a predetermined threshold level;
a membrane valve extending across the bypass channel, the membrane valve having an opening therethrough sized to tightly receive therein and seal around a power injection device; and
a flapper valve extending across the bypass channel distal of the membrane valve, the flapper valve including a flap biased toward a closed position sealing the bypass channel and openable by the power injection device.

8. The valved catheter according to claim 7, wherein the flapper valve includes a hinge pivotally connecting the flap to a peripheral portion of the flapper valve, the peripheral portion being coupled to the housing.

9. The valved catheter according to claim 7, wherein the distal opening of the bypass channel is distal of the distal opening of the infusion channel.

10. The valved catheter according to claim 7, wherein the flapper valve is formed as a check valve opening only toward the distal opening of the bypass channel.

11. The valved catheter according to claim 7, wherein the elongate body is a peripherally inserted central catheter.

12. The bypass according to claim 7, wherein the flapper valve is formed of a resilient material biasing the flap toward the closed position.

13. The bypass according to claim 7, wherein the flapper valve includes a biasing member biasing the flap toward the closed position.

14. A method for transferring fluids to the vascular system, comprising:
transferring fluid through an infusion/withdrawal channel of a catheter at a first flow rate, the infusion/withdrawal channel including a pressure activated safety valve sealing the infusion/withdrawal channel at all times when a fluid pressure within the infusion/withdrawal channel is less than a predetermined threshold level;
inserting a power injection device into a bypass channel of the catheter, through a hole in a membrane valve to push open and pass through a flapper valve, the hole in the membrane being sized to tightly receive and seal around an outer surface of the power injection device; and
power injecting fluid into the catheter via the bypass channel at a second flow rate higher than the first flow rate.

15. The method according to claim 14, further comprising withdrawing the power injection device from the bypass channel, withdrawal of the power injection device allowing the flapper valve to move to a sealed position toward which it is biased.
